(19) United States
(12) Patent Application Publication
(10) Pub. No.: US 2006/0014815 A1
(43) Pub. Date: Jan. 19, 2006

(54) Process for making pyrazole compounds

(76) Inventors: Toshiaki Mase, Okazaki (JP); Takehiko Iida, Okazaki (JP); Masashi Kawasaki, Okazaki (JP); Kenichi Asakawa, Okazaki (JP)

Correspondence Address: WENDEROTH, LIND & PONACK, L.L.P., 2033 K STREET N. W., SUITE 800, WASHINGTON, DC, 20006-1021, US

(21) Appl. No.: 10/531,317

(22) PCT Filed: Oct. 22, 2003
(86) PCT No.: PCT/JP03/13507

§ 371 Date: May 23, 2005

Related U.S. Application Data
(60) Provisional application No. 60/420,590, filed on Oct. 23, 2002.

(51) Int. Cl. A61K 31/415; C07D 231/14

(52) U.S. Cl. 514/406; 548/371.4

(57) ABSTRACT

This invention relates to a process for making pyrazole compounds of formula I.

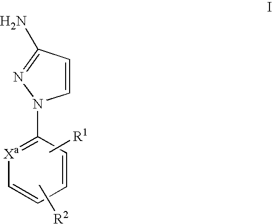

### TECHNICAL FIELD

The present invention provides a process for preparing compounds of structural formula I

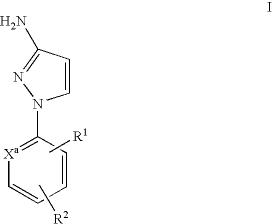

The process involves converting an unsubstituted or substituted phenyl hydrazine salt, or an unsubstituted or substituted pyridine hydrazine salt, of formula III, such as the hydrochloride salt IIIA, into the free phenyl hydrazine III′, or the free pyridyl hydrazine III, with a base. Alternatively, the process may start with the free phenyl hydrazine III′, or the free pyridyl hydrazine III. The free phenyl hydrazine III′, or the free pyridyl hydrazine III, is then reacted with an acrylonitrile to form the unsubstituted or substituted phenyl pyrazole, or unsubstituted or substituted pyridyl pyrazole, of formula I. The pyrazole of formula I may be treated with an acid to form the pyrazole salt of general formula IC, wherein X^{a} is CH, CR¹, CR² or nitrogen.

Scheme A illustrates the preparation of pyrazoles of formula I, and salts thereof as exemplified by IC, wherein X^{a} is CH, CR¹, CR² or nitrogen.

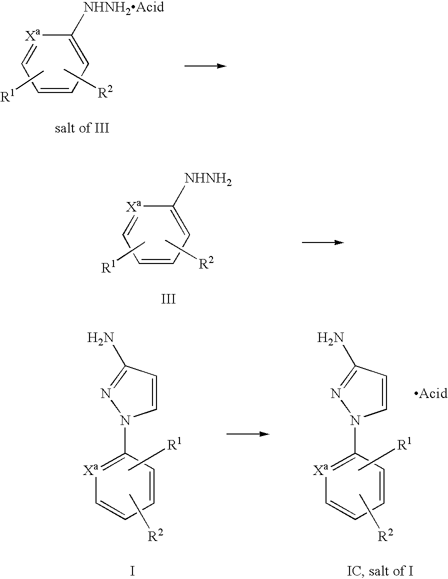

Reacting the pyrazole I, or the pyrazole salt IC, with a spirolactone of formula IV gives spirolactone amides of general structural formula II.

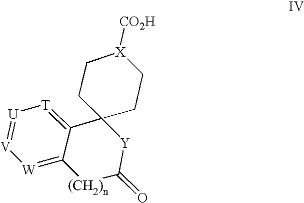

### BACKGROUND ART

The present invention relates to a process for the preparation of the pyrazole of formula I.

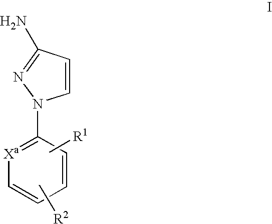

The compounds of formula I are intermediates useful for the preparation of the spirolactone compounds of formula II.

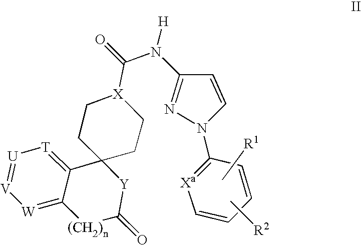

The compounds of formula II, along with their use as NPY5 antagonists for treating bulimia, obesity or diabetes, were disclosed in U.S. Pat. No. 6,335,345, which is incorporated by reference herein in its entirety, and in WO 01/14376 (published on Mar. 02, 2001). The compounds of formula II are also useful as agents for the treatment of various diseases related to NPY, including, but not limited to, cardiovascular disorders, such as hypertension, nephropathy, heart disease, vasospasm, arteriosclerosis and the like, central nervous system disorders, such as bulimia, depression, anxiety, seizure, epilepsy, dementia, pain, alcoholism, drug withdrawal and the like, metabolic diseases such as obesity, diabetes, hormone abnormality, hypercholesterolemia, hyperlipidemia and the like, sexual and reproductive dysfunction, gastrointestinal disorder, respiratory disorder, inflammation or glaucoma, and the like.

U.S. Pat. No. 6,335,345, which is incorporated by reference herein in its entirety, and WO 01/14376, describe a process for preparing the compounds of formula II.

Processes for the preparation of 1-phenylpyrazol-3-amine by reacting a phenylhydrazine with 2-chloro-acrylonitrile, 3-chloroacrylonitrile, 2,3-dichloro-propanenitrile, or 2,3-dibromopropanenitrile are described in the Journal of Heterocyclic Chemistry, vol. 19, pp. 1265 and 1267 (1982). However, for the reactions utilizing 2-chloroacrylonitrile, 2,3-dichloropropanenitrile, and 2,3-dibromopropanenitrile, the yield of the 1-phenylpyrazol-3-amine is very low. Additionally, the 3-chloroacrylonitrile starting material is very difficult to prepare.

### DISCLOSURE OF INVENTION

By this invention, there is provided a process for the preparation of a compound of structural formula I′, or a salt, hydrate or polymorph thereof, 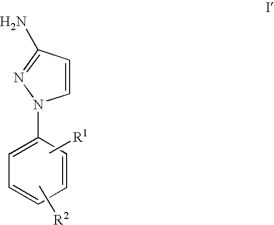
wherein R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
   comprising the steps of:
(a) forming a hydrazine solution;
(b) adding a compound of formula V 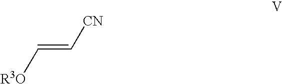
   wherein R³ is selected from the group consisting of
(1) lower alkyl,
(2) aryl, and
(3) —CH₂aryl,
   to the hydrazine solution of step (a) to form a mixture; and
(c) heating the mixture of step (b) to a temperature between about 50° C. to about 100° C.;
to afford the compound I′, or a salt, hydrate or polymorph thereof.

In one embodiment of the present invention, the hydrazine solution of step (a) is formed by dissolving a compound of formula III′ 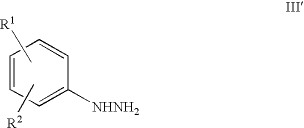
in a solvent.

In one class of this embodiment, the solvent is selected from the group consisting of
(a) C₁₋₄ alcohol;
(b) toluene;
(c) tetrahydrofuran; and
(d) dimethylformamide,
   or a mixture thereof.

In one subclass of this class, the solvent is ethanol. In another subclass, the solvent is toluene-ethanol.

In another embodiment of the present invention, the hydrazine solution of step (a) is formed by treating a salt of a compound of formula III′ 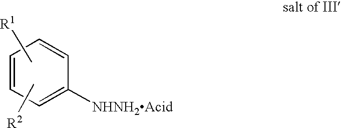
with a base in a solvent.

In one class of this embodiment, the solvent is selected from the group consisting of
(a) C₁₋₄ alcohol;
(b) toluene;
(c) tetrahydrofuran; and
(d) dimethylformamide,
   or a mixture thereof.

In a subclass of this class, the solvent is ethanol. In another subclass of this class, the solvent is toluene-ethanol or tert-butanol.

In another class of this embodiment, the salt of the compound of formula III′ is selected from the group consisting of hydrochloride salt, hydrobromide salt, dihydrobromide salt, mesylate salt, tosylate salt, besylate salt and sulfate salt. In a subclass of this class, the salt of the compound of formula III′ is a hydrochloride salt.

In another class of this embodiment, the base is selected from the group consisting of
(a) sodium ethoxide,
(b) sodium methoxide,
(c) lower alkylamine,
(d) 1,8-diazabicyclo[5.4.0]undec-7-ene,
(e) potassium t-butoxide, and
(f) sodium hydroxide.

In a subclass of this class, the base is sodium ethoxide.

In another embodiment, R³ is selected from the group consisting of lower alkyl. In a class of this embodiment, R³ is selected from the group consisting of: —CH₃, —CH₂CH₃, —(CH₂) ₂CH₃, —CH(CH₃)₂, —(CH₂)₃CH₃, and —C(CH₃)₃. In a subclass of this class, R³ is —CH₂CH₃.

In another embodiment of the present invention, in the step (b) the amount of the compound of the formula V relative to that of a hydrazine is preferably about 0.8 to 1.8 in terms of molar ratio.

In another embodiment of the present invention, step (c) is aged for a period of about 2 hours to 48 hours, preferably about 4 hours to 48 hours. In a class of this embodiment, step (c) is aged for a period of about 2 to 30 hours, preferably about 10 to 30 hours.

In another embodiment of this invention, the process further comprises step (d) of isolating the compound of formula I′.

In another embodiment of this invention, R¹ and R² are independently selected from the group consisting of
(1) hydrogen,
(2) halogen,
(3) lower alkyl,
(4) halo(lower)alkyl,
(5) lower alkenyl,
(6) lower alkanoyl,
(7) lower alkylene optionally substituted with oxo, and
(8) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

In a class of this embodiment, R¹ is hydrogen and R² is selected from the group consisting of
(1) hydrogen,
(2) 2-fluoro,
(3) 3-fluoro,
(4) 4-fluoro,
(5) 5-fluoro,
(6) 2-chloro,
(7) 3-chloro,
(8) 4-chloro,
(9) 2-difluoromethoxy,
(10) 3-difluoromethoxy,
(11) 2-methyl,
(12) 2-pyridyl,
(13) 2-quinolyl, and
(14) 3-quinolyl.

In a subclass of this class, R¹ is hydrogen and R² is selected from the group consisting of
(1) hydrogen,
(2) 2-fluoro,
(3) 3-fluoro, and
(4) 4-fluoro.

In another subclass of this class, both R¹ and R² are hydrogen.

In another subclass of this class, R¹ is hydrogen and R² is 2-fluoro.

In yet another subclass of this class, R¹ is hydrogen and R² is 4-fluoro.

In another embodiment of this invention, the process further comprises the step (e) of treating the compound of formula I′ 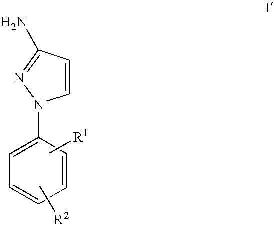
with an acid to form a salt.

In one class of this embodiment, the acid of step (e) is selected from the group consisting of acetic acid, oxalic acid, hydrobromic acid, hydrochloric acid, anhydrous p-toluene-sulfonic acid, p-toluenesulfonic acid hydrate, p-toluene-sulfonic acid monohydrate, benzenesulfonic acid, and methanesulfonic acid, or a mixture thereof.

In one subclass of this class, the acid of step (e) is selected from the group consisting of acetic acid, oxalic acid, hydrochloric acid, anhydrous p-toluenesulfonic acid, p-toluenesulfonic acid hydrate, p-toluenesulfonic acid monohydrate, and benzenesulfonic acid or mixture thereof.

In another subclass of this class, the acid of step (e) is hydrochloric acid.

In yet another subclass of this class, the acid of step (e) is p-toluenesulfonic acid monohydrate.

In another class of this embodiment, the salt formed is the p-toluenesulfonic acid salt of formula IA′, or a hydrate or polymorph thereof, 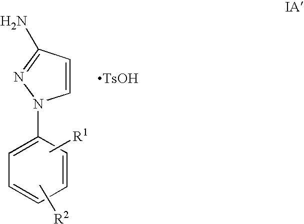
wherein R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

In yet another class of this embodiment, the salt formed is the hydrochloric acid salt of formula IB′, or a hydrate or polymorph thereof, 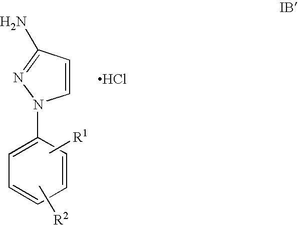
wherein R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

By this invention, there is also provided a compound of formula IA′ 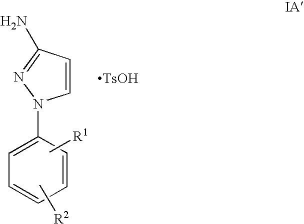
wherein R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl,
   or a hydrate or polymorph thereof.

By this invention, there is also provided a compound of formula IB′ 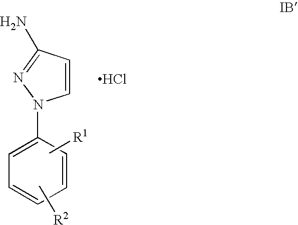
wherein R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl,
   or a hydrate or polymorph thereof.

By this invention, there is also provided a process for the preparation of a compound of structural formula I, or a salt, hydrate or polymorph thereof, 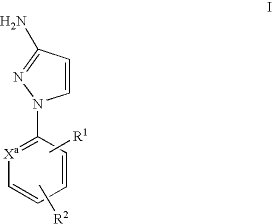
wherein
X^{a} is CH, CR¹, CR² or nitrogen;
R¹ and R² are both independently selected from the group consisting of
   (1) hydrogen,
   (2) halogen,
   (3) nitro,
   (4) lower alkyl,
   (5) halo(lower)alkyl,
   (6) hydroxy(lower)alkyl,
   (7) cyclo(lower)alkyl,
   (8) lower alkenyl,
   (9) lower alkoxy,
   (10) halo(lower)alkoxy,
   (11) lower alkylthio,
   (12) carboxyl,
   (13) lower alkanoyl,
   (14) lower alkoxycarbonyl,
   (15) lower alkylene optionally substituted with oxo, and
   (16) -Q-Ar², wherein Q is selected from the group consisting of a single bond and a carbonyl, and
      wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
   comprising the steps of:
(a) forming a hydrazine solution;
(b) adding a compound of formula V 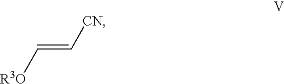
   wherein
R³ is selected from the group consisting of
   (1) lower alkyl,
   (2) aryl, and
   (3) —CH₂aryl,
to the hydrazine solution of step (a) to form a mixture; and
(c) heating the mixture of step (b) to a temperature between about 50° C. to about 100° C.;
to afford the compound I, or a salt, hydrate or polymorph thereof.

In one embodiment of the present invention, the hydrazine solution of step (a) is formed by dissolving a compound of formula III 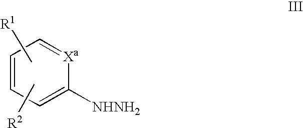
in a solvent.

In one class of this embodiment, the solvent is selected from the group consisting of
(a) C₁₋₄ alcohol;
(b) toluene;
(c) tetrahydrofuran; and
(d) dimethylformamide,
   or a mixture thereof.

In one subclass of this class, the solvent is ethanol. In another subclass, the solvent is tert-butanol or toluene-ethanol.

In another embodiment of the present invention, the hydrazine solution of step (a) is formed by treating a salt of a compound of formula III, 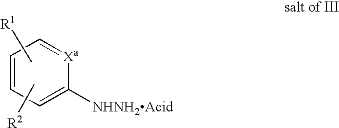
with a base in a solvent.

In one class of this embodiment, the solvent is selected from the group consisting of
(a) C₁₋₄ alcohol;
(b) toluene;
(c) tetrahydrofuran; and
(d) dimethylformamide,
   or a mixture thereof.

In a subclass of this class, the solvent is ethanol. In another subclass of this class, the solvent is tert-butanol.

In another class of this embodiment, the base is selected from the group consisting of
(a) sodium ethoxide,
(b) sodium methoxide,
(c) lower alkylamine,
(d) 1,8-diazabicyclo[5.4.0]undec-7-ene,
(e) potassium t-butoxide, and
(f) sodium hydroxide.

In a subclass of this class, the base is potassium tert-butoxide.

In another class of this embodiment, the salt of the compound of formula III is selected from the group consisting of hydrochloride salt, hydrobromide salt, dihydrobromide salt, mesylate salt, tosylate salt, besylate salt and sulfate salt. In a subclass of this class, the salt of the compound of formula III is a hydrochloride salt.

In another embodiment, R³ is selected from the group consisting of lower alkyl. In a class of this embodiment, R³ is selected from the group consisting of: —CH₃, —CH₂CH₃, —(CH₂)₂CH₃, —CH(CH₃)₂, —(CH₂)₃CH₃, and —C(CH₃)₃. In a subclass of this class, R³ is —CH₂CH₃.

In another embodiment of the present inventions in the step (b) the amount of the compound of the formula V relative to that of a hydrazine is preferably about 0.8 to 1.8 in terms of molar ratio.

In another embodiment of the present invention, step (c) is aged for a period of about 2 hours to 48 hours. In a class of this embodiment, step (c) is aged for a period of about 2 to 5 hours.

In another embodiment of this invention, the process further comprises step (d) of isolating the compound of formula I.

In another embodiment of this invention, R¹ and R² are independently selected from the group consisting of
(1) hydrogen,
(2) halogen,
(3) lower alkyl,
(4) halo(lower)alkyl,
(5) lower alkenyl,
(6) lower alkanoyl,
(7) lower alkylene optionally substituted with oxo, and
(8) -Q-Ar², wherein Q is selected from the group consisting of a single bond and a carbonyl, and
   wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

In a class of this embodiment, R¹ is hydrogen and R² is selected from the group consisting of
(1) hydrogen,
(2) 2-fluoro,
(3) 3-fluoro,
(4) 4-fluoro,
(5) 5-fluoro,
(6) 2-chloro,
(7) 3-chloro,
(8) 4-chloro,
(9) 2-difluoromethoxy,
(10) 3-difluoromethoxy,
(11) 2-methyl,
(12) 2-pyridyl,
(13) 2-quinolyl, and
(14) 3-quinolyl.

In a subclass of this class, R¹ is hydrogen and R² is selected from the group consisting of
(1) hydrogen,
(2) 2-fluoro,
(3) 3-fluoro, and
(4) 4-fluoro.

In another subclass of this class, both R¹ and R² are hydrogen.

In another subclass of this class, R¹ is hydrogen and R² is 2-fluoro.

In yet another subclass of this class, R¹ is hydrogen and R² is 4-fluoro.

In another embodiment of this invention, the process further comprises the step (e) of treating the compound of formula I 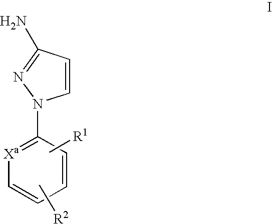
with an acid to form a salt.

In one class of this embodiment, the acid of step (e) is selected from the group consisting of acetic acid, oxalic acid, hydrobromic acid, hydrochloric acid, anhydrous p-toluene-sulfonic acid, p-toluenesulfonic acid hydrate, p-toluene-sulfonic acid monohydrate, benzenesulfonic acid, and methane sulfonic acid, or a mixture thereof.

In one subclass of this class, the acid of step (e) is selected from the group consisting of acetic acid, oxalic acid, hydrochloric acid, anhydrous p-toluenesulfonic acid, p-toluene-sulfonic acid hydrate, p-toluenesulfonic acid monohydrate and benzenesulfonic acid, or a mixture thereof.

In another subclass of this class, the acid of step (e) is hydrochloric acid.

In yet another subclass of this class, the acid of step (e) is p-toluene sulfonic acid monohydrate.

In another class of this embodiment, the salt formed is the p-toluenesulfonic acid salt of formula IA, or a hydrate or polymorph thereof, 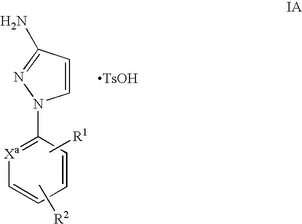
wherein
X^{a} is CH, CR¹, CR² or nitrogen;
R¹ and R² are both independently selected from the group consisting of
   (1) hydrogen,
   (2) halogen,
   (3) nitro,
   (4) lower alkyl,
   (5) halo(lower)alkyl,
   (6) hydroxy(lower)alkyl,
   (7) cyclo(lower)alkyl,
   (8) lower alkenyl,
   (9) lower alkoxy,
   (10) halo(lower)alkoxy,
   (11) lower alkylthio,
   (12) carboxyl,
   (13) lower alkanoyl,
   (14) lower alkoxycarbonyl,
   (15) lower alkylene optionally substituted with oxo, and
   (16) -Q-Ar², wherein Q is selected from the group consisting of a single bond and a carbonyl, and
      wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

In yet another class of this embodiment, the salt formed is the hydrochloric acid salt of formula IB, or a hydrate or polymorph thereof, 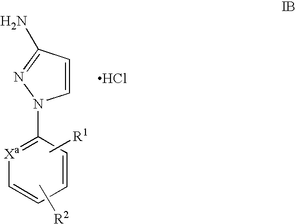
wherein
X^{a} is CH, CR¹, CR² or nitrogen;
R¹ and R² are both independently selected from the group consisting of
   (1) hydrogen,
   (2) halogen,
   (3) nitro,
   (4) lower alkyl,
   (5) halo(lower)alkyl,
   (6) hydroxy(lower)alkyl,
   (7) cyclo(lower)alkyl,
   (8) lower alkenyl,
   (9) lower alkoxy,
   (10) halo(lower)alkoxy,
   (11) lower alkylthio,
   (12) carboxyl,
   (13) lower alkanoyl,
   (14) lower alkoxycarbonyl,
   (15) lower alkylene optionally substituted with oxo, and
   (16)-Q-Ar², wherein Q is selected from the group consisting of a single bond and a carbonyl, and
wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

By this invention, there is also provided a compound of formula IA 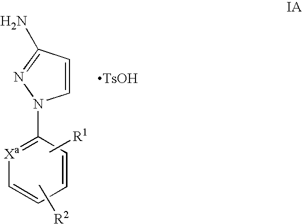
wherein
X^{a} is CH, CR¹, CR² or nitrogen;
R¹ and R² are both independently selected from the group consisting of
   (1) hydrogen,
   (2) halogen,
   (3) nitro,
   (4) lower alkyl,
   (5) halo(lower)alkyl,
   (6) hydroxy(lower)alkyl,
   (7) cyclo(lower)alkyl,
   (8) lower alkenyl,
   (9) lower alkoxy,
   (10) halo(lower)alkoxy,
   (11) lower alkylthio,
   (12) carboxyl,
   (13) lower alkanoyl,
   (14) lower alkoxycarbonyl,
   (15) lower alkylene optionally substituted with oxo, and
   (16)-Q-Ar², wherein Q is selected from the group consisting of a single bond and a carbonyl, and
      wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl,
   or a hydrate or polymorph thereof.

By this invention, there is also provided a compound of formula IB 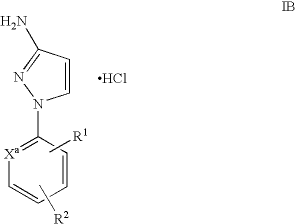
wherein
X^{a} is CH, CR¹, CR² or nitrogen;
R¹ and R² are both independently selected from the group consisting of
   (1) hydrogen,
   (2) halogen,
   (3) nitro,
   (4) lower alkyl,
   (5) halo(lower)alkyl,
   (6) hydroxy(lower)alkyl,
   (7) cyclo(lower)alkyl,
   (8) lower alkenyl,
   (9) lower alkoxy,
   (10) halo(lower)alkoxy,
   (11) lower alkylthio,
   (12) carboxyl,
   (13) lower alkanoyl,
   (14) lower alkoxycarbonyl,
   (15) lower alkylene optionally substituted with oxo, and
   (16) -Q-Ar², wherein Q is selected from the group consisting of a single bond and a carbonyl, and
      wherein Ar² is selected from the group consisting of
(1) aryl, and
(2) heteroaryl,
   wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl,
   or a hydrate or polymorph thereof.

By this invention, there is also provided a compound of formula 1-3 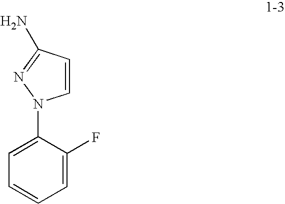
or a hydrate or polymorph thereof.

By this invention, there is also provided a compound of formula 1-4 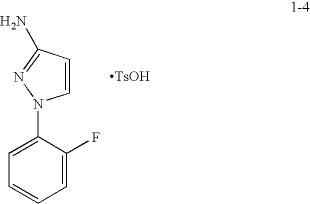
or a hydrate or polymorph thereof.

By this invention, there is also provided a crystalline form of the tosylate salt of compound 1-4

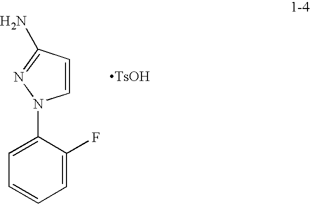

By this invention, there is also provided a compound of 2-1 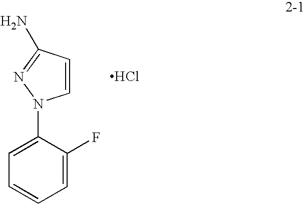
or a hydrate or polymorph thereof.

By this invention, there is also provided a compound which is a crystalline form of the hydrochloride salt of compound 2-1

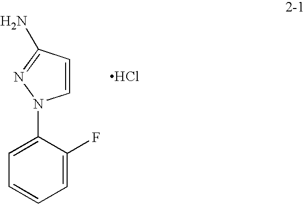

The compounds in the processes of the present invention include stereoisomers, such as optical isomers, diastereomers and geometrical isomers, or tautomers depending on the mode of substitution. The present invention is meant to comprehend all such isomeric forms of the compounds in the compositions of the present invention, and their mixtures. All hydrates, solvates and polymorphic crystalline forms of the above-described compounds and their use, including their use in the processes of the instant invention, are encompassed within scope of the instant invention.

“Halogen” refers to fluorine atom, chlorine atom, bromine atom and iodine atom.

“C₁₋₄ alcohol” refers to methanol, ethanol, n-propanol, isopropanol, n-butanol, isobutanol, sec-butanol and tert-butanol, and the like.

“Lower alkyl” refers to a straight- or branched-chain alkyl group of C₁ to C₆, for example, methyl, ethyl, propyl, isopropyl, butyl, isobutyl, sec-butyl, tert-butyl, pentyl, isopentyl, hexyl, isohexyl, and the like.

“Halo(lower)alkyl” refers to the aforesaid lower alkyl substituted with 1 or more than 2, preferably 1 to 3 aforesaid halogen atoms identically or differently at the substitutable, arbitrary positions, for example, fluoromethyl, difluoromethyl, trifluoromethyl, 2-fluoroethyl, 1,2-difluoroethyl, chloromethyl, 2-chloroethyl, 1,2-dichloroethyl, bromomethyl, iodomethyl, and the like.

“Hydroxy(lower) alkyl” refers to the aforesaid lower alkyl substituted with 1 or more than 2, preferably 1 or 2 hydroxy groups at the substitutable, arbitrary positions, for example, hydroxymethyl, 2-hydroxyethyl, 1-hydroxy-1-methylethyl, 1,2-dihydroxyethyl, 3-hydroxypropyl, and the like.

“Cyclo(lower)alkyl” refers to a cycloalkyl group of C₃ to C₆, for example, cyclopropyl, cyclobutyl, cyclopentyl, cyclohexyl, and the like.

“Lower alkenyl” refers to a straight- or branched-chain alkenyl group of C₂ to C₆, for example, vinyl, 1-propenyl, 2-propenyl, isopropenyl, 3-butenyl, 2-butenyl, 1-butenyl, 1-methyl-2-propenyl, 1-methyl-1-propenyl, 1-ethyl-1-ethenyl, 2-methyl-2-propenyl, 2-methyl-1-propenyl, 3-methyl-2-butenyl, 4-pentenyl, and the like.

“Lower alkoxy” refers to a straight- or branched-chain alkoxy group of C₁ to C₆, for example, methoxy, ethoxy, propoxy, isopropoxy, butoxy, sec-butoxy, isobutoxy, tert-butoxy, pentyloxy, isopentyloxy, hexyloxy, isohexyloxy, and the like.

“Halo(lower)alkoxy” refers to the aforesaid lower alkoxy substituted with 1 or more than 2, preferably 1 to 3 aforesaid halogen atoms identically or differently at the substitutable, arbitrary positions, for example, fluoromethoxy, difluoromethoxy, trifluoromethoxy, 2-fluoroethoxy, 1,2-difluoroethoxy, chloromethoxy, 2-chloroethoxy, 1,2-dichloroethoxy, bromomethoxy, iodomethoxy, and the like.

“Lower alkylthio” refers to a straight- or branched-chain alkylthio group of C₁ to C₆, for example, methylthio, ethylthio, propylthio, isopropylthio, butylthio, sec-butylthio, isobutylthio, tert-butylthio, pentylthio, isopentylthio, hexylthio, isohexylthio, and the like.

“Lower alkylamine” refers to an amine which is mono-, di- or trisubstituted with a straight- or branched-chain alkyl group of C₁ to C₄, for example, methylamine, ethylamine, propylamine, isopropylamine, butylamine, sec-butylamine, isobutylamine, tert-butylamine, dimethyl amine, trimethyl amine, diethyl amine, triethyl amine, diisopropylethyl amine, and the like.

“Lower alkanoyl” refers to an alkanoyl group containing the aforesaid lower alkyl, that is, an alkanoyl group of C₂ to C₇, for example acetyl, propionyl, butyryl, isobutyryl, valeryl, isovaleryl, pivaloyl, and the like.

“Lower alkoxycarbonyl” refers to an alkoxycarbonyl group containing the aforesaid lower alkoxy, that is, an alkoxycarbonyl group of C₂ to C₇, for example, methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, and the like.

“Lower alkylene optionally substituted with oxo” refers to a straight- or branched-chain alkylene group of C₂ to C₆ which may be substituted with 1 or more than 2, preferably 1 oxo group at a substitutable, arbitrary position, for example, ethylene, trimethylene, tetramethylene, pentamethylene, hexamethylene, 1-oxoethylene, 1-oxotrimethylene, 2-oxotrimethylene, 1-oxotetramethylene, 2-oxotetramethylene, and the like. The above alkylene group is formed by combining R¹ and R², taken together.

“Aryl” includes phenyl, naphthyl, and the like.

“Heteroaryl” refers to 5- or 6-membered monocylic heteroaromatic group which contains 1 or more than 2, preferably 1 to 3 hetero atoms identically or differently selected from the group consisting of oxygen atom, nitrogen atom and sulfur atom; or condensed heteroaromatic group, where the aforesaid monocylic heteroaromatic group is condensed with the aforesaid aryl group, or with the identified or different aforesaid monocylic heteroaromatic group each other, for example, pyrrolyl, furyl, thienyl, imidazolyl, pyrazolyl, thiazolyl, isothiazolyl, oxazolyl, isoxazolyl, 1,2,3-triazolyl, 1,2,4-triazolyl, tetrazolyl, oxadiazolyl, 1,2,3-thiadiazolyl, 1,2,4-thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzoxazolyl, benzisoxazolyl, benzothiazolyl, benzisothiazolyl, indazolyl, purinyl, quinolyl, isoquinolyl, phthalazyl, naphthylidinyl, quinoxalinyl, quinazolinyl, cinnolinyl, pteridinyl, pyrido[3,2-b]pyridyl, and the like.

“Lower alkylamino” refers to an amino group mono-substituted with the aforesaid lower alkyl, for example, methylamino, ethylamino, propylamino, isopropylamino, butylamino, sec-butylamino, tert-butylamino, and the like.

“Di-lower alkylamino” refers to an amino group di-substituted with identical or different aforesaid lower alkyl, for example, dimethylamino, diethylamino, ethylmethylamino, dipropylamino, methylpropylamino, diisopropylamino, and the like.

In order to disclose the aforesaid compounds of the general formula I more detailed, the various symbols used in the formula I are explained in more detail by the use of preferred embodiments.

“Aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar²” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or more than 2, preferably 1 or 2 selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group of formula: -Q-Ar².

Halogen atom as the aforesaid substituent includes, preferably, fluorine atom, chlorine atom, and the like.

Lower alkyl as the aforesaid substituent includes, preferably, methyl, ethyl, propyl, isopropyl, and the like.

Halo(lower)alkyl as the aforesaid substituent includes, preferably, difluoromethyl, trifluoromethyl, and the like.

Hydroxy(lower)alkyl as the aforesaid substituent includes, preferably, hydroxymethyl, 2-hydroxyethyl, 1-hydroxy-1-methylethyl, and the like.

Cyclo(lower)alkyl as the aforesaid substituent includes, preferably, cyclopropyl, cyclobutyl, and the like.

Lower alkenyl as the aforesaid substituent includes, preferably, vinyl, 1-propenyl, 2-methyl-1-propenyl, and the like.

Lower alkoxy as the aforesaid substituent includes, preferably, methoxy, ethoxy, and the like.

Halo(lower)alkoxy as the aforesaid substituents includes, preferably, fluoromethoxy, difluoromethoxy, trifluoromethoxy, and the like.

Lower alkylthio as the aforesaid substituent includes, preferably, methylthio, ethylthio, and the like.

Lower alkanoyl as the aforesaid substituent includes, preferably, acetyl, propionyl, and the like.

Lower alkoxycarbonyl as the aforesaid substituent includes, preferably, methoxycarbonyl, ethoxycarbonyl, and the like.

Lower alkylene optionally substituted with oxo as the aforesaid substituent includes, preferably, 1-oxotetramethylene, and the like.

In a group of formula: -Q-Ar² as the aforesaid substituent, Ar² represents aryl or heteroaryl which may be substituted, the substituent being selected from the group consisting of halogen, cyano, lower alkyl, halo(lower)alkyl, hydroxy(lower)alkyl, hydroxy, lower alkoxy, halo (lower) alkoxy, lower alkylamino, di-lower alkylamino, lower alkanoyl and aryl; Q represents a single bond or carbonyl.

“Aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or not less than 2, preferably 1 or 2 selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl.

Halogen atom as the aforesaid substituent includes, preferably, fluorine atom, chlorine atom, and the like.

Lower alkyl as the aforesaid substituent includes, preferably, methyl, ethyl, propyl, isopropyl, and the like.

Halo(lower)alkyl as the aforesaid substituent includes, preferably, difluoromethyl, trifluoromethyl, and the like.

Hydroxy(lower)alkyl as the aforesaid substituent includes, preferably, hydroxymethyl, 2-hydroxyethyl, 1-hydroxy-1-methylethyl, and the like.

Lower alkoxy as the aforesaid substituent includes, preferably, methoxy, ethoxy, and the like.

Halo(lower)alkoxy as the aforesaid substituent includes, preferably, fluoromethoxy, difluoromethoxy, trifluoromethoxy, and the like.

Lower alkylamino as the aforesaid substituent includes, preferably, methylamino, ethylamino, and the like.

Di-lower alkylamino as the aforesaid substituent includes, preferably, dimethylamino, diethylamino, and the like.

Lower alkanoyl as the aforesaid substituent includes, preferably, acetyl, propionyl, and the like.

Aryl as the aforesaid substituent includes, preferably, phenyl, and the like.

The substituent(s) of Ar² include, preferably, halogen, cyano, lower alkyl, halo(lower)alkyl, hydroxy(lower)alkyl, hydroxy, halo(lower)alkoxy, and the like.

Aryl in Ar² includes, preferably, phenyl, and the like and heteroaryl includes imidazolyl, pyridyl, benzofuranyl, quinolyl, and the like.

Consequently, a group of formula: -Q-Ar² includes, for example, phenyl, 2-fluorophenyl, 3-fluorophenyl, 4-fluorophenyl, 2,3-difluorophenyl, 2,4-difluorophenyl, 3,5-difluorophenyl, 2-chlorophenyl, 3-chlorophenyl, 4-chlorophenyl, 2-cyanophenyl, 3-cyanophenyl, 4-cyanophenyl, 2-methylphenyl, 3-methylphenyl, 4-methylphenyl, 2-fluoro-5-methylphenyl, 3-fluoromethylphenyl, 2-trifluoromethylphenyl, 3-trifluoromethylphenyl, 4-trifluoromethylphenyl, 2-methoxyphenyl, 3-methoxyphenyl, 4-methoxyphenyl, 3-fluoro-5-methoxyphenyl, 3-fluoromethoxyphenyl, 3-difluoromethoxyphenyl, 3-(2-hydroxyethyl)phenyl, 3-hydroxymethylphenyl, 3-(1-hydroxy-1-methylethyl)phenyl, 3-hydroxyphenyl, 4-hydroxyphenyl, 2-imidazolyl, 1-ethyl-2-imidazolyl, 1,2,4-thiadiazol-5-yl, 1,3,4-thiadiaol-2-yl, 2-pyridyl, 3-pyridyl, 4-pyridyl, 2-ethyl-4-pyridyl, 4-pyrimidinyl, 5-pyrimidinyl, 4-benzo[b]furanyl, 5-benzo[b]furanyl, 7-benzo[b]furanyl, 2-quinolyl, 3-quinolyl, 4-quinolyl, 5-quinolyl, 6-quinolyl, 8-quinolyl, benzoyl, 2-pyridylcarbonyl, and the like, and preferably, phenyl, 2-fluorophenyl, 3-fluorophenyl, 3,5-difluorophenyl, 3-chlorophenyl, 4-chlorophenyl, 3-cyanophenyl, 3-trifluoromethylphenyl, 3-difluoromethoxyphenyl, 3-(2-hydroxyethyl)phenyl, 3-hydroxyphenyl, 4-hydroxyphenyl, 1-ethyl-2-imidazolyl, 2-pyridyl, 7-benzo[b]furanyl, 2-quinolyl, 3-quinolyl, benzoyl, 2-pyridylcarbonyl, and the like.

The salts of compounds of formula I, including, but not limited to, compounds of formula IA, IB, and IC, refer to the pharmaceutically acceptable and common salts, for example, base addition salt to carboxyl group when the compound has a carboxyl group, or acid addition salt to amino or basic heterocyclyl when the compound has an amino or basic heterocyclyl group, and the like.

The base addition salts include salts with alkali metals (including, but not limited to, sodium, potassium); alkaline earthmetals (including, but not limited to, calcium, magnesium); ammonium or organic amines (including, but not limited to, trimethylamine, triethylamine, dicyclohexylamine, ethanolamine, diethanolamine, triethanolamine, procaine, N,N′-dibenzylethylenediamine), and the like.

The acid addition salts include salts with inorganic acids (including, but not limited to, hydrochloric acid, sulfuric acid, nitric acid, phosphoric acid, perchloric acid), organic acids (including, but not limited to, acetic acid, oxalic acid, maleic acid, fumaric acid, tartaric acid, citric acid, ascorbic acid, trifluoroacetic acid, acetic acid), sulfonic acids (including, but not limited to, methanesulfonic acid, isethionic acid, benzenesulfonic acid, p-toluenesulfonic acid, p-toluenesulfonic acid monohydrate, p-toluenesulfonic acid hydrate, camphor sulfonic acid), and the like.

Polymorphism can be defined as the ability of the same chemical substance to exist in different crystalline structures. The different structures are referred to as polymorphs, polymorphic modifications or forms. The pyrazole tosylate salt 1-4 has been found it exist in at least two polymorphic nonsolvated forms, Form A and Form B, each of which can be formed by careful control of the crystallization conditions.

In the schemes and examples below, various reagent symbols and abbreviations have the following meanings:
AcOEt or EtOAc: ethyl acetate
tBuOH: tert-butanol
tert-BuOH: tert-butanol
DBU: 1,8-diazabicyclo[5.4.0]undec-7-ene
EtOH: ethanol
g: grams
IPAC: isopropyl acetate
HCl: hydrochloric acid
HPLC: high pressure liquid chromatography
KOtBu: potassium tert-butoxide
NaCl: sodium chloride
NaHCO₃: sodium bicarbonate
NaOEt: sodium ethoxide
NaOH: sodium hydroxide
mL: milliliter
mmol: millimole
mol: moles/liter
MTBE: methyl t-butyl ether
THF: tetrahydrofuran
TsOH: p-toluenesulfonic acid
TsOH.H₂O: p-toluenesulfonic acid monohydrate

The compounds of the present invention can be prepared by employing the following General Scheme, which shows one embodiment of the present invention wherein a 2-fluorophenyl-hydrazine salt of compound III is reacted with an acrylonitrile of formula V. The pyrazole compounds of formula I, and salts and polymorphs thereof, are prepared from commercially available starting materials, such as 2-fluorophenylhydrazine hydrochloride 1-1, and ethoxyacrylonitrile 1-2, as shown in Example 1 and 2.

### EXAMPLES

The following examples are provided to illustrate the invention and are not to be construed as limiting the scope of the invention in any manner.

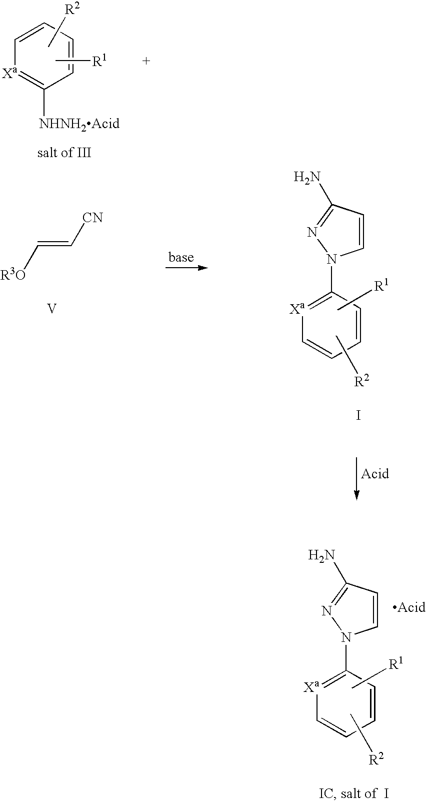

### Example 1

### Preparation of 1-(2-Fluorophenyl)-1H-Pyrazole-3-Amine Tosylate 1-4

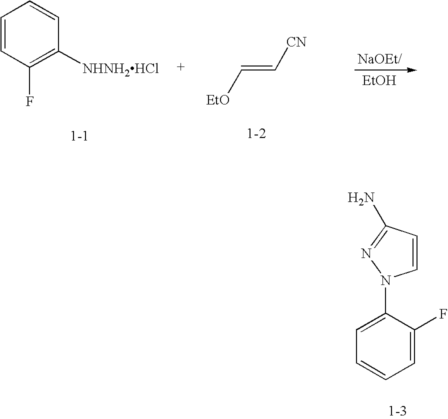

### Step A: Preparation of 1-(2-Fluorophenyl)-1H-Pyrazole-3-Amine 1-3

To a suspension of the 2-fluorophenylhydrazine hydrochloride 1-1 (50 g, JEMCO, Inc.) in EtOH (300 mL) was added 20 weight % NaOEt in EtOH (292.97 g, Nihon Soda). The ethoxyacrylonitrile 1-2 (53.76 g, Degussa) was then added at ambient temperature. The reaction mixture was warmed to about 82° C. and aged for 20 to 28 hours. The reaction mixture was cooled to ambient temperature. To the batch was added water (250 mL, 5 volumes) and 6N HCl to adjust the mixture to a pH between about 2.9-3.1. The resulting aqueous EtOH solution was stirred at 20° C. to 25° C. for 1 to 2 hours. After treatment with 5N NaOH to adjust the solution to a pH of about 6.5 to 8.0, the reaction mixture was concentrated to circa 600 mL (12 volumes), then IPAC (750 mL) was added. The layers were separated and the organic layer was washed with 10% aqueous NaCl (200 mL). Activated carbon (Sirasagi P, 1.75 g, 3.5 weigh % to 2-fluorophenylhydrazine HCl) was added to the resulting solution at ambient temperature. After 1 to 20 hours treatment of the activated carbon, the cake was washed with IPAC (4 volumes to a weight % to 2-fluorophenylhydrazine HCl, 200 mL). The combined organic layers were concentrated to about 410-510 mL (10-12.5 volumes to assay gram of pyrazole 1-3) to give 1-(2-fluorophenyl)-1H-pyrazole-3-amine 1-3. Selected Signals ¹H NMR (300 MHz, DMSO-d₆): δ 7.84 (d, J=2.6 Hz, 1H), 7.72 (dd, J=8.2, 1.8 Hz, 1H), 7.34 (ddd, J=11.1, 7.9, 1.7 Hz, 1H), 7.28-7.14 (m, 2H), 5.77 (d, J=2.6 Hz, 1H), 5.10 (brs, 2H).

Compound 1-3 is also characterized by differential scanning calorimetry (DSC). The DSC curve for compound 1-3 is characterized by an endotherm with a peak temperature of 46.98° C.+2° C., when obtained under the following measurement conditions:
Appratus: DSC 2920(TA Instruments)
Sample cell: 60 microliter Hasteroy B closed cell
(KASEN Engineering Co., Ltd.)
Lamp: 10° C./min. (ambient—300° C.)
Atmosphere:
in cell: atomospheric pressure
out cell: atomospheric pressure.

### Step B: Preparation of the Tosylate Salt 1-4

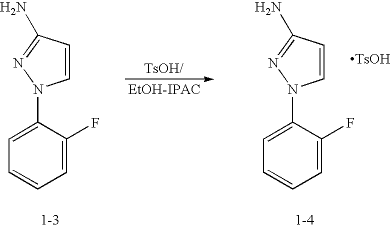

Pyrazole tosylate (0.5 weight % to assay grams of pyrazole, 105 mg, form-II) was added to the reaction mixture as seed. TsOH.H₂O (27.07 g 142.32 mmol, 1.2 equivalents to assay % of pyrazole 1-3) in EtOH (67.2 mL) was added to the solution of compound 1-3, from step A, over 3 hours, followed by IPAC (2.5 volumes to assay grams of pyrazole, 52.5 mL) over 1 hour at room tempdderature. The mixture was stirred for about 14 to 17 hours. The batch was cooled to 0° C., aged for 2 hours and then filtered. The cake was washed with EtOH-IPAC (1:9, 84 mL), IPAC (84 mL), and then dried in vacuo at 30° C. to give the pyrazole tosylate salt 1-4 (Form-II crystal).

Selected Signals: ¹H NMR (500 MHz, DMSO-d₆): δ 9.68 (brs, 3H), 8.24 (dd, J=2.0, 2.0 Hz, 1H), 7.72 (dd, J=8.0, 8.0 Hz, 1H), 7.51-7.42 (m, 4H), 7.37 (dd, J=7.6, 7.6 Hz, 1H), 7.12 (d, J=7.9 Hz, 2H), 6.44 (d, J=2.3 Hz, 1H), 2.28 (s, 3H)^{˜}

Instead of seeding form-II crystals, form-I crystal seeding and the above treatment gave the form-I crystal of pyrazole tosylate.

Crystal Form-I

The prepared 1-(2-fluorophenyl)-1H-pyrazole-3-amine tosylate salt 1-4 (Form-II crystal, 1 g) was stirred in EtOH-MTBE (1:4.5 mixture, 20.1 mL) at room temperature for 23 hours. The crystal was filtered and washed with MTBE to give 1-(2-fluorophenyl)-1H-pyrazole-3-amine tosylate salt 1-4 (Form-I crystal, 95%).

Crystal Form-II

To a solution of crude 1-(2-fluorophenyl)-1H-pyrazole-3-amine 1-3 (3.42 g, 18.29 mmol.) in EtOH (13.7 mL) was added p-toluenesulfonic acid (4.41 g, 23.2 mmol.) in EtOH (11 mL), and then dropwise MTBE (8.6 mL) over 0.5 h at room temperature. The seed (pyrazole tosylate, form I crystal, 0.25 weight % to assay grams of pyrazole) was added then aged at this temperature for 0.5 h. To this slurry was added additional MTBE (103 mL) over 3.0 hours and stirred for 13 hours at room temperature. The crystal was filtered and washed with MTBE-EtOH (9:1, 27.4 mL) to give 1-(2-fluorophenyl)-1H-pyrazole-3-amine tosylate salt 1-4 (Form-II crystal, 58%).

The following powder X-ray diffraction analysis data in Tables 1, 2 and 3 were measured by RINT1100 (manufactured by Rigaku International Corporation) and analysis methods were as follows:
X-ray radiation source: Cu,
tube voltage: 40 KV,
tube current: 30 mA,
monochronomater: automatic monochromater
monoreceiving slit: 0.60 mm
goniometer: wide angle goniometer,
scan step: 0.02 degrees,
scan speed: 2.00 degrees/minute,
divergence slit (DS): 1 degree,
scattering slit: 1 degree,
receiving slit (RS): 0.15 millimeter,
measured temperature: ambient temperature.
   Table 1. Powder X-ray diffraction:

### 1-(2-Fluorophenyl)-1H-Pyrazole-3-Amine Tosylate 1-4, Crystal Form-I

| 2θ(2 theta)(degrees) | Intensity(cps) |
|---|---|
| 5.020 | 573 |
| 7.700 | 183 |
| 9.400 | 617 |
| 9.600 | 642 |
| 13.300 | 116 |
| 14.240 | 2230 |
| 14.500 | 973 |
| 14.660 | 2589 |
| 14.920 | 140 |
| 15.400 | 262 |
| 15.900 | 2225 |
| 16.020 | 2582 |
| 17.140 | 198 |
| 19.180 | 805 |
| 19.460 | 1358 |
| 20.020 | 6311 |
| 21.360 | 476 |
| 21.680 | 1705 |
| 22.840 | 1142 |
| 23.000 | 1575 |
| 23.140 | 928 |
| 23.640 | 834 |
| 24.540 | 343 |
| 25.340 | 263 |
| 25.620 | 2769 |
| 25.700 | 3756 |
| 25.980 | 773 |
| 26.460 | 545 |
| 26.680 | 611 |
| 26.980 | 558 |
| 27.420 | 279 |
| 28.200 | 1494 |
| 28.740 | 123 |
| 29.460 | 450 |
| 30.020 | 256 |
| 30.580 | 124 |
| 31.240 | 2024 |
| 31.520 | 309 |
| 31.900 | 253 |
| 32.300 | 233 |
| 33.620 | 305 |
| 34.820 | 254 |
| 35.260 | 343 |
| 35.860 | 163 |
| 36.300 | 159 |
| 37.260 | 123 |
| 37.680 | 219 |
| 38.220 | 204 |
| 38.700 | 231 |
| 39.060 | 173 |

Although Form I of 1-(2-fluorophenyl)-1H-pyrazole-3-amine tosylate 1-4 is characterized by the complete group of angle 2 theta values listed in Table 1, all the values are not required for such indentification. Form I of 1-(2-fluorophenyl)-1H-pyrazole-3-amine tosylate 1-4 can be identified by the angle theta value in the range of 14.2 to 14.3°. Form I of 1-(2-fluorophenyl)-1H-pyrazole-3-amine tosylate 1-4 can be identified by any one of the following angle theta values, or any one of the following groups of angle theta values:
a) 14.24°;
b) 14.2-14.3° and 21.6-21.7°;
c) 14.2-14.3°, 20.0-20.1°, and 21.6-21.7°;
d) 14.2-14.3°, 20.0-20.1°, 21.6-21.7°, and 31.2-31.3°;

e) 14.24°, 14.6-14.7°, 15.9°, 16.0-16.1°, 19.4-19.5°, 20.0-20.1°, 21.6-21.7°, 22.8-22.9°, 23°, 25.6-25.7°, 25.7°, 28.2° and 31.2-31.3°. Additionally, each of the angle 2 theta values from Table 1 can be expressed to two decimal places as follows: 14.24°, 14.66°, 15.90°, 16.02°, 19.46°, 20.02°, 21.68°, 22.84°, 23.00°, 25.62°, 25.70°, 28.20° and 31.24°.

TABLE 2
Powder X-ray diffraction:
1-(2-Fluorophenyl)-1H-Pyrazole-3-Amine Tosylate 1-4, Crystal Form-II

| 2θ(2 theta)(degrees) | Intensity(cps) |
|---|---|
| 2.220 | 384 |
| 8.680 | 4040 |
| 9.500 | 395 |
| 11.980 | 3610 |
| 14.560 | 276 |
| 15.340 | 1130 |
| 15.680 | 238 |
| 16.080 | 129 |
| 16.720 | 206 |
| 17.460 | 190 |
| 17.780 | 272 |
| 18.200 | 726 |
| 18.820 | 1295 |
| 19.160 | 211 |
| 20.100 | 565 |
| 20.520 | 3939 |
| 20.660 | 2817 |
| 22.500 | 1494 |
| 23.640 | 398 |
| 24.040 | 196 |
| 24.420 | 239 |
| 24.920 | 889 |
| 25.740 | 214 |
| 26.080 | 504 |
| 26.360 | 808 |
| 27.100 | 288 |
| 28.240 | 1106 |
| 29.320 | 234 |
| 29.880 | 581 |
| 30.280 | 310 |
| 30.920 | 267 |
| 32.940 | 376 |
| 34.280 | 159 |
| 34.700 | 358 |
| 35.420 | 146 |
| 37.140 | 161 |
| 37.440 | 199 |
| 38.360 | 248 |
| 38.940 | 398 |
| 39.680 | 209 |

Although Form II of 1-(2-fluorophenyl)-1H-pyrazole-3-amine tosylate 1-4 is characterized by the complete group of angle 2 theta values listed in Table 2, all the values are not required for such indentification. Form II of 1-(2-fluorophenyl)-1H-pyrazole-3-amine tosylate 1-4 can be identified by the angle theta value in the range of 8.6 to 8.7°. Form II of 1-(2-fluorophenyl)-1H-pyrazole-3-amine tosylate 1-4 can be identified by any one of the following angle theta values, or any one of the following groups of angle theta values:
a) 8.68°;
b) 8.6-8.7° and 11.9-12.0°;
c) 8.6-8.7°, 11.9-12.0°, and 20.5-20.6°;
d) 8.6-8.7°, 11.9-12.0°, 20.5-20.6°, and 20.6-20.7°; and
e) 8.6-8.7°, 11.9-12.0°, 15.3-15.4°, 18.8-18.9°, 20.5-20.6°, 20.6-20.7°, and 22.5°. Additionally, each of the angle 2 theta values from Table 1 can be expressed to two decimal places as follows: 8.68°, 11.98°, 15.34°, 18.82°, 20.52°, 20.66°, 22.50°, and 28.24°.

Compound 1-4 is also characterized by differential scanning calorimetry (DSC). The DSC curve for compound 1-3 is characterized by an endotherm with a peak temperature of 140.29° C.+2° C., when obtained under the same measurement conditions as for compound 1-3, Example 1, Step A.

### Example 2

### Preparation of 1-(2-Fluorophenyl)-1H-Pyrazole-3-Amine Hydrochloride 2-1

### Step A: Preparation of 1-(2-Fluorophenyl)-1H-Pyrazole-3-Amine 1-3

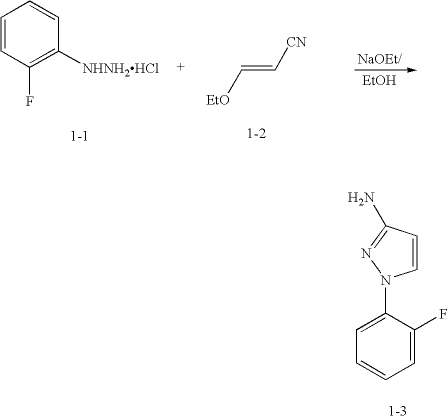

To a suspension of the 2-fluorophenylhydrazine hydrochloride 1-1 (12.5 g, 76.9 mmol, JEMCO) in EtOH (75 mL, 6 volumes) was added 20 weight % NaOEt in EtOH (72.9 g) while keeping the temperature less than 30° C. The ethoxyacrylonitrile 1-2 (13.4 g, Degussa) was then added at 25° C. The reaction mixture was warmed to about 82° C. over 30 minutes and then aged for 20 to 28 hours. The reaction mixture was cooled to ambient temperature. Water (62.5 mL, 5 volumes) and 6N HCl, to adjust the mixture to a pH between 2.9 to 3.1, were slowly added to the reaction mixture while keeping the temperature below 30° C. The resulting aqueous ethanol solution was stirred at a temperature of about 20° C. to 25° C. for 1 to 2 hours, then treated with 5N NaOH, to adjust the pH to between 6.5 to 8.0. The resulting solution was concentrated to 150 mL (12 volumes) in vacuo at 40° C., and then extracted with toluene (125 mL) two times.

The organic layer was washed with 10% aqueous NaCl (62.5 mL, 5 volumes). Activated carbon (Shirasagi P, 3.5 weight % to 2-fluorophenylhydrazine HCl, 473.5 mg) was added to the resulting solution at ambient temperature and stirred for about 15 to 20 hours. The cake (activated carbon) was washed with toluene (4 volumes to assay grams of pyrazole, 40.9 mL). The washings were combined with the filtrate to give 1-(2-fluorophenyl)-1H-pyrazole-3-amine 1-3.

Selected Signals: ¹H NMR (300 MHz, DMSO-d₆): δ 7.84 (d, J=2.6 Hz, 1H), 7.72 (dd, J=8.2, 1.8 Hz, 1H), 7.34 (ddd, J=11.1, 7.9, 1.7 Hz, 1H), 7.28-7.14 (m, 2H), 5.77 (d, J=2.6 Hz, 1H), 5.10 (brs, 2H).

### Step B: Preparation of the Hydrochloride Salt 2-1

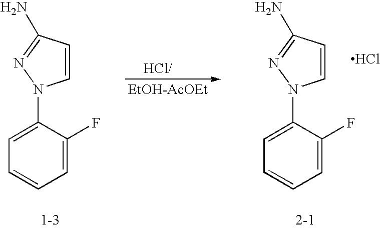

A portion of the above organic layer containing 1-(2-fluorophenyl)-1H-pyrazole-3-amine 1-3 (115 mL, 51.0 mg/mL, 5.87 assay g (33.13 mmol)) was solvent-switched from toluene to EtOH (29.4 mL, 5 volumes to pyrazole assay). To the solution was added EtOAc (5.9 mL, 1 volume to assay grams of pyrazole), followed by 4N HCl in EtOAc (9.11 mL, 36.4 mmol, 1.1 equivalents) at room temperature. Then the 1-(2-fluorophenyl)-1H-pyrazole-3-amine HCl salt (0.5 weight % to assay grams of pyrazole, 29.4 mg) was added as seed.

The resulting slurry was aged at room temperature for 1 hour, and then EtOAc (88 mL, 15 volumes to pyrazole assay) was added dropwise at ambient temperature over more than 2 hours. The resulting suspension was aged at ambient temperature for 15 to 20 hours. The batch was filtered, washed with EtOH-AcOEt (1:10; 23.5 mL), EtOAc (11.7 mL), and dried at room temperature under vacuum for 15 hours to give the 1-(2-fluorophenyl)-1H-pyrazole-3-amine hydrochloride salt 2-1.

Selected Signals ¹H NMR (500 MHz, DMSO-d₆): δ 9.18 (brs, 3H), 8.20.(dd, J=2.4, 2.4 Hz, 1H), 7.73.(ddd, J=8.0, 8.0, 1.6.Hz, 1H), 7.50-7.42.(m, 2H), 7.36.(ddd, J=8.0, 8.0, 1.5 Hz, 1H), 6.40.(d, J=2.5 Hz, 1H)^{˜}

### Powder X-ray diffraction: 1-(2-Fluorophenyl)-1H-Pyrazole-3-Amine HCl Salt 2-1

| 2θ(2 theta)(degrees) | Intensity(cps) |
|---|---|
| 10.580 | 242 |
| 10.920 | 1187 |
| 11.740 | 489 |
| 14.880 | 377 |
| 17.660 | 874 |
| 19.020 | 192 |
| 19.400 | 1254 |
| 19.940 | 2149 |
| 22.080 | 1911 |
| 22.560 | 390 |
| 22.820 | 705 |
| 23.140 | 640 |
| 23.680 | 1771 |
| 24.160 | 405 |
| 24.680 | 2102 |
| 26.500 | 134 |
| 27.060 | 518 |
| 27.600 | 1539 |
| 28.260 | 286 |
| 29.140 | 844 |
| 29.860 | 476 |
| 31.340 | 534 |
| 32.360 | 588 |
| 32.900 | 169 |
| 33.320 | 204 |
| 33.700 | 400 |
| 34.860 | 795 |
| 35.460 | 136 |
| 35.820 | 225 |
| 36.760 | 150 |
| 37.400 | 357 |
| 37.740 | 177 |
| 38.340 | 150 |
| 39.380 | 379 |

Above powder X-ray diffraction analysis data were measured by the same conditions as Example 1 (Step B).

Although 1-(2-fluorophenyl)-1H-pyrazole-3-amine hydrochloride salt 2-1 is characterized by the complete group of angle 2 theta values listed in Table 3, all the values are not required for such indentification. The 1-(2-fluorophenyl)-1H-pyrazole-3-amine hydrochloride salt 2-1 can be identified by the angle theta value in the range of 19.9-20.0°. The 1-(2-fluorophenyl)-1H-pyrazole-3-amine hydrochloride salt 2-1 can be identified by any one of the following angle theta values, or any one of the following groups of angle theta values:
a) 19.94°;
b) 10.9-11.0°, 19.9-20.0°, and 24.6-24.7°; and
c) 10.9-11.0°, 19.4°, 19.9-20.0°, 22.0-22.1°, 23.6-23.7°, 24.6-24.7° and 27.6°. Additionally, each of the angle 2 theta values from Table 1 can be expressed to two decimal places as follows: 10.92°, 19.40°, 19.94°, 22.08°, 23.68°, 24.68° and 27.60°.

Compound 2-1 is also characterized by differential scanning calorimetry (DSC). The DSC curve for compound 1-3 is characterized by an endotherm with a peak temperature of 145.65° C.+2° C., when obtained under the same measurement conditions as for compound 1-3, Example 1, Step A.

### Example 3

### Preparation of 1-(2-Phenyl)-1H-Pyrazole-3-Amine 3-2

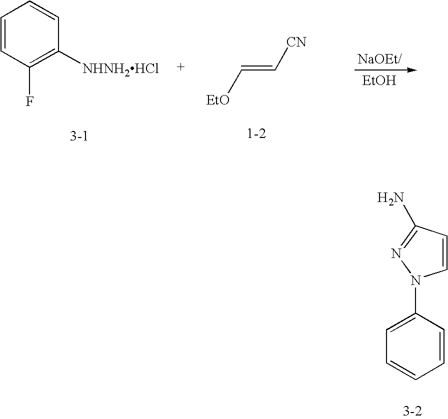

To a suspension of the phenylhydrazine hydrochloride 3-1 (1.0 g, TCI) in EtOH (5 mL) was added 21 weight % NaOEt in EtOH (7.23 mL) while keeping the temperature less than 30° C. The ethoxyacrylonitrile 1-2 (1.33 mL, Acros) was then added at 25° C. The reaction mixture was warmed to about 82° C. over 30 minutes and then aged for 20 hours. The reaction mixture was cooled to ambient temperature. Water (10 mL) was slowly added to the reaction mixture while keeping the temperature below 30° C. The resulting aqueous ethanol solution was extraced with MTBE (20 mL) then the organic layer was washed with 10% NaCl aqueous solution (5 mL). Activated carbon (Shirasagi P, 5 mg) was added to the resulting solution at ambient temperature and stirred for about 1 hour. Concentration of the filtrate and purification of the resulting residue with flash chromatography (heptane/EtOAc=2:1) gave 1-(2-Phenyl)-1H-Pyrazole-3-Amine 3-2.

¹HNMR (500 MHz, DMSO-d₆): δ 8.12 (d, J=2.5 Hz, 1H), 7.63 (d, J=8.3 Hz, 2H), 7.38 (dd, J=7.9, 7.9 Hz, 2H), 7.11 (dd, J=7.3, 7.3 Hz, 1H), 5.73 (d, J=2.5 Hz, 1H), 5.06 (brs, 2H)

Alternatively, 1-phenyl-1H-pyrazole-3-amine 3-2 may also be prepared according to the synthethic procedure shown in Example 4.

### Example 4

### Preparation of 1-Phenyl-1H-Pyrazole-3-Amine 3-2

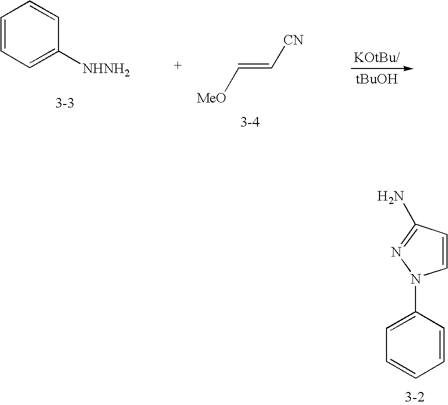

To a hot solution of tert-BuOK (100 g, Tokyo Kasei) in tert-BuOH (650 mL) was added phenylhydrazine 3-3 (39.36 mL, Tokyo Kasei). After cooling to ambient temperature, methoxyacrylonitrile 3-4 (33.57 mL, Tokyo Kasei) was added dropwise and the mixture was refluxed for 15 hours. The reaction mixture was cooled to ambient temperature and the solvent was removed by evaporation. To the residue was added water (200 mL) and EtOAc (500 mL). The layers were separated and the organic layer was washed with brine (200 mL), dried over MgSO₄ and concentrated. To the residue was added 5N HCl (200 mL) and EtOAc (500 mL) and the precipitated solids were removed by filtration. The filtered layers were separated, and the organic layer was extracted with 5N HCl (100 mL). The aqueous layers were combined and treated with 5N NaOH to adjust the solution to a pH of about 9, then the aqueous solution was extracted with EtOAc (400 mL+200 mL). The organic layers were combined and washed with brine (100 mL), dried over MgSO₄ and concentrated. The resulting residue was purified by flash chromatography on silica gel (Wako gel C-300, Wako, EtOAc/hexane 1:9 to 1:1) to give compound 3-2.

¹H NMR (300 MHz, DMSO-d₆): δ 8.11 (d, J=2.6 Hz, 1H), 7.62 (dd, J=8.7, 1.1 Hz, 2H), 7.37 (dd, J=8.7, 7.4 Hz, 2H), 7.10 (dt, J=7.4, 1.1 Hz, 1H), 5.72 (d, J=2.6 Hz, 1H), 5.01 (brs, 2H).

### Example 5

### 5 Preparation of 1-(2-Pyridyl)-1H-Pyrazole-3-Amine 5-3

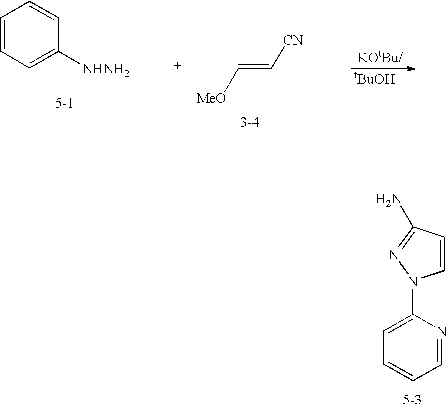

To a hot solution of tert-BuOK (2.7 g, Tokyo Kasei) in tert-BuOH (60 mL) was added 2-hydrazinopyridine 5-1 (2.18 g, Aldrich). After cooling to ambient temperature, a solution of methoxyacrylonitrile 3-4 (1.68 mL, Tokyo Kasei) in tert-BuOH (10 mL) was added and the reaction mixture was refluxed for 3 hours. The reaction mixture was cooled to ambient temperature and the solvent was removed by evaporation. To the residue was added water and EtOAc. The layers were separated and the organic layer was washed with brine, dried over Na₂SO₄ and concentrated. The residue was purified by flash chromatography on silica gel (Wako gel C-300, Wako, EtOAc/hexane 1:2 to 1:1) to give compound 5-3.

¹H NMR (300 MHz, CDCl₃): δ 8.35-8.29 (m, 2H), 7.75-7.68 (m, 2H), 7.09-7.01 (m, 1H), 5.88-5.83 (m, 1H), 3.89 (brs, 2H).

The following 1H-pyrazole-3-amines were prepared by the same procedure using corresponding hydrazine or its hydrochloride (supplied by Tokyo Kasei Kogyo, Wako Pure Chemicals, Kanto Chemicals, Aldrich Chemical Company or Lancaster Synthesis).

### 1-(3,4-Dichlorophenyl)-1H-Pyrazole-3-Amine

¹HNMR (300 MHz, DMSO-d₆): δ 8.22 (s, 1H), 7.90 (s, 1H), 7.70-7.55 (m, 2H), 5.80 (s, 1H), 5.22 (brs, 2H)

### 1-(2-Methoxyphenyl)-1H-Pyrazole-3-Amine

¹H NMR (300 MHz, DMSO-d₆): δ 7.90-7.80 (m, 1H), 7.70-7.60 (m, 1H), 7.50-6.80 (m, 3H), 5.85-5.70(m, 1H), 3.98 (s, 3H)

### 1-(2-Methylphenyl)-1H-Pyrazole-3-Amine

¹H NMR (200 MHz, CDCl₃): δ 7.35 (d, J=2.4 Hz, 1H), 7.22-7.19 (m, 4H), 5.81 (d, J=2.4 Hz, 1H), 3.9 (brs, 2H), 2.29 (s, 3H)

### 1-(3-Fluorophenyl)-1H-Pyrazole-3-Amine

¹H NMR (200 MHz, CDCl₃): δ 7.68 (d, J=2.6 Hz, 1H), 7.39-7.28 (m, 3H), 6.91-6.79 (m, 1H), 5.86 (d, J=2.6 Hz, 1H), 3.82 (brs, 2H)

### 1-(4-Cyanophenyl)-1H-Pyrazole-3-Amine

¹H NMR (300 MHz, DMSO-d₆): δ 8.28 (d, J=2.7 Hz, 1H), 7.85-7.75 (m, 4H), 5.84 (d, J=2.7 Hz, 1H), 5.31 (brs, 2H)

### 1-(4-Chlorophenyl)-1H-Pyrazole-3-Amine

¹H NMR (300 MHz, CDCl₃): δ 7.64 (d, J=2.7 Hz, 1H), 7.55-7.42 (m, 2H), 7.40-7.29 (m, 2H), 5.85 (d, J=2.7 Hz, 1H), 3.82 (brs, 2H)

### 1-(3-Chlorophenyl)-1H-Pyrazole-3-Amine

¹H NMR (300 MHz, CDCl₃): δ 7.67 (d, J=2.6 Hz, 1H), 7.65-7.50 (m, 1H), 7.46-7.39 (m, 1H), 7.33-7.24 (m, 1H), 7.17-7.11 (m, 1H), 5.84 (d, J=2.6 Hz, 1H), 3.82 (brs, 2H)

### 1-(2,4-Difluorophenyl)-1H-Pyrazole-3-Amine

¹H NMR (200 MHz, CDCl₃): δ 7.84-7.69 (m, 2H), 7.00-6.87 (m, 2H), 5.87 (d, J=2.6 Hz, 1H), 3.85 (brs, 2H)

### 1-(3,5-Difluorophenyl)-1H-Pyrazole-3-Amine

¹H NMR (300 MHz, CDCl₃): δ 7.64 (d, J=2.6 Hz, 1H), 7.17-7.06 (m, 2H), 6.63-6.55 (m, 1H), 5.88 (d, J=2.6 Hz, 1H), 3.86 (brs, 2H)

### 1-(4-Fluorophenyl)-1H-Pyrazole-3-Amine

¹H NMR (200 MHz, CDCl₃): δ 7.64-7.43 (m, 3H), 7.16-7.00 (m, 2H), 5.83 (d, J=2.5 Hz, 1H), 3.84 (brs, 2H).

Employing the procedure substantially as described in Examples 1, 2, 3, 4 or 5, but substituting the appropriate amines for the 2-fluorophenylhydrazine and phenyl hydrazine starting materials used in these Examples, other substituted pyrazole compounds of formula I may be prepared.

While the invention has been described and illustrated with reference to certain particular embodiments thereof, those skilled in the art will appreciate that various changes, modifications and substitutions can be made therein without departing from the spirit and scope of the invention. It is intended, therefore, that the invention be defined by the scope of the claims which follow and that such claims be interpreted as broadly as is reasonable.

### INDUSTRIAL APPLICABILITY

The present invention relates to a process for the preparation of the pyrazole of formula I.

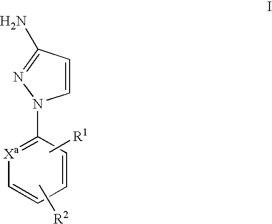

The compounds of formula I are intermediates useful for the preparation of the spirolactone compounds of formula II.

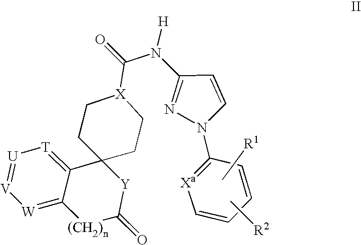

The compounds of formula II are also useful as agents for the treatment of various diseases related to NPY, including, but not limited to, cardiovascular disorders, such as hypertension, nephropathy, heart disease, vasospasm, arteriosclerosis and the like, central nervous system disorders, such as bulimia, depression, anxiety, seizure, epilepsy, dementia, pain, alcoholism, drug withdrawal and the like, metabolic diseases such as obesity, diabetes, hormone abnormality, hypercholesterolemia, hyperlipidemia and the like, sexual and reproductive dysfunction, gastrointestinal disorder, respiratory disorder, inflammation or glaucoma, and the like.

1. A process for preparing a compound of the formula I′, or a salt, hydrate or polymorph thereof, 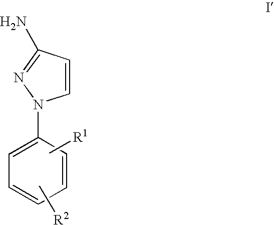
wherein
R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
wherein Ar² is selected from the group consisting of
(1) aryl, and
(2) heteroaryl,
wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
comprising the steps of:
(a) forming a hydrazine solution;
(b) adding a compound of formula V 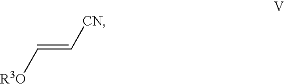
wherein
R³ is selected from the group consisting of
(1) lower alkyl,
(2) aryl, and
(3) —CH₂aryl,
to the hydrazine solution of step (a) to form a mixture; and
(c) heating the mixture of step (b) to a temperature between about 50° C. to about 100° C.;
to afford the compound I′, or a salt, hydrate or polymorph thereof.

2. The process of claim 1 wherein the hydrazine solution of step (a) is formed by dissolving a compound of formula III′ 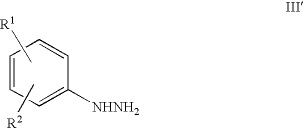
in a solvent.

3. The process of claim 2, wherein the solvent is selected from the group consisting of
(a) C₁₋₄ alcohol;
(b) toluene;
(c) tetrahydrofuran; and
(d) dimethylformamide;
or a mixture thereof.

4. The process of claim 3 wherein the solvent is ethanol.

5. The process of claim 1 wherein the hydrazine solution of step (a) is formed by treating a salt of a compound of formula III′ 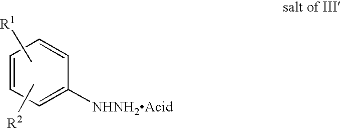
with a base in a solvent.

6. The process of claim 5 wherein the solvent is selected from the group consisting of
(a) C₁₋₄ alcohol;
(b) toluene;
(c) tetrahydrofuran; and
(d) dimethylformamide;
or a mixture thereof.

7. The process of claim 6 wherein the solvent is ethanol.

8. The process of claim 5 wherein the salt of the compound of formula III′ is selected from the group consisting of acetic acid salt, oxalic acid salt, hydrochloride salt, hydrobromide salt, dihydrobromide salt, mesylate salt, tosylate salt, besylate salt and sulfate salt.

9. The process of claim 8 wherein the salt of the compound of formula III′ is a hydrochloride salt.

10. The process of claim 5 wherein the base is selected from the group consisting of
(a) sodium ethoxide;
(b) sodium methoxide;
(c) lower alkylamine;
(d) 1,8-diazabicyclo[5.4.0]undec-7-ene;
(e) potassium t-butoxide; and
(f) sodium hydroxide.

11. The process of claim 10 wherein the base is sodium ethoxide.

12. The process of claim 1 wherein R¹ and R² are both independently selected from the group consisting of
(1) hydrogen,
(2) halogen,
(3) lower alkyl,
(4) halo(lower)alkyl,
(5) lower alkenyl,
(6) lower alkanoyl,
(7) lower alkylene, optionally substituted with oxo, and
(8) -Q-Ar², wherein Q is selected from the group consisting of a single bond and a carbonyl, and
wherein Ar² is selected from the group consisting of
(1) aryl, and
(2) heteroaryl,
wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl.

13. The process of claim 12 wherein R¹ is hydrogen and R² is selected from the group consisting of
(1) hydrogen,
(2) 2-fluoro,
(3) 3-fluoro,
(4) 4-fluoro,
(5) 5-fluoro,
(6) 2-chloro,
(7) 3-chloro,
(8) 4-chloro,
(9) 2-difluoromethoxy,
(10) 3-difluoromethoxy,
(11) 2-methyl,
(12) 2-pyridyl,
(13) 2-quinolyl, and
(14) 3-quinolyl.

14. The process of claim 13 wherein R¹ is hydrogen and R² is selected from the group consisting of
(1) hydrogen,
(2) 2-fluoro,
(3) 3-fluoro, and
(4) 4-fluoro.

15. The process of claim 14 wherein both R¹ and R² are hydrogen.

16. The process of claim 14 wherein R¹ is hydrogen and R² is 2-fluoro.

17. The process of claim 14 wherein R¹ is hydrogen and R² is 4-fluoro.

18. The process of claim 1 wherein R³ is selected from the group consisting of lower alkyl.

19. The process of claim 18 wherein R³ is selected from the group consisting of: —CH₃, —CH₂CH₃, —(CH₂)₂CH₃, —CH(CH₃)₂, —(CH₂)₃CH₃, and —C(CH₃)₃.

20. The process of claim 19 wherein R³ is —CH₂CH₃.

21. The process of claim 1 further comprising the step (d) of isolating the compound I′.

22. The process of claim 1 further comprising the step (e) of treating compound I′ with an acid to form a salt.

23. The process of claim 22 wherein the acid of step (e) is selected from the group consisting of acetic acid, oxalic acid, hydrobromic acid, hydrochloric acid, anhydrous p-toluene-sulfonic acid, p-toluenesulfonic acid hydrate, p-toluene-sulfonic acid monohydrate, benzenesulfonic acid, and methanesulfonic acid, or a mixture thereof.

24. The process of claim 23 wherein the acid of step (e) is selected from the group consisting of acetic acid, oxalic acid, hydrochloric acid, anhydrous p-toluenesulfonic acid, p-toluene-sulfonic acid hydrate, benzenesulfonic acid, and p-toluenesulfonic acid monohydrate, or a mixture thereof.

25. The process of claim 24 wherein the acid of step (e) is p-toluenesulfonic acid monohydrate.

26. The process of claim 24 wherein the acid of step (e) is hydrochloric acid.

27. A compound of formula 1-4 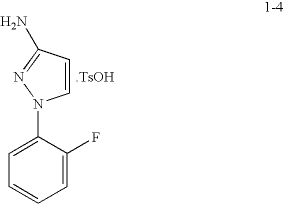
or a hydrate or polymorph thereof.

28. A compound which is a crystalline form of the tosylate salt of compound 1-4

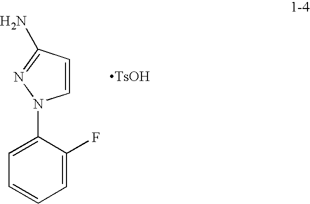

29. The compound of claim 28 having an x-ray powder diffraction pattern obtained using Cu radiation containing an angle 2 theta value of 14.2-14.3°.

30. The compound of claim 28 having an x-ray powder diffraction pattern obtained using Cu radiation containing an angle 2 theta value of 14.24°.

31. The compound of claim 28 having an x-ray powder diffraction pattern obtained using Cu radiation containing the following angle 2 theta values: 14.2-14.3°and 21.6-21.7°.

32. The compound of claim 28 having an x-ray powder diffraction pattern obtained using Cu radiation containing the following angle 2 theta values: 14.2-14.3°, 20.0-20.1°, and 21.6-21.7°.

33. The compound of claim 28 having an x-ray powder diffraction pattern obtained using Cu radiation containing an angle 2 theta value of 8.6-8.7°.

34. The compound of claim 28 having an x-ray powder diffraction pattern obtained using Cu radiation containing an angle 2 theta value of 8.68°.

35. The compound of claim 28 having an x-ray powder diffraction pattern obtained using Cu radiation containing the following angle 2 theta values:
8.6-8.7° and 11.9-12.0°.

36. The compound of claim 28 having an x-ray powder diffraction pattern obtained using Cu radiation containing the following angle 2 theta values:
8.6-8.7°, 11.9-12.0°, and 20.5-20.6°.

37. A compound of formula 2-1 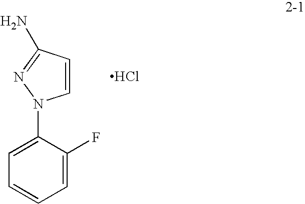
or a hydrate or polymorph thereof.

38. A compound which is a crystalline form of the hydrochloride salt of compound 2-1

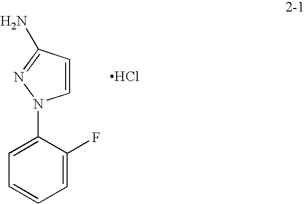

39. The compound of claim 38 having an x-ray powder diffraction pattern obtained using Cu radiation containing the following angle 2 theta value:
19.9-20.0°.

40. The compound of claim 38 having an x-ray powder diffraction pattern obtained using Cu radiation containing the following angle 2 theta value:
19.94°.

41. The compound of claim 38 having an x-ray powder diffraction pattern obtained using Cu radiation containing the following angle 2 theta values:
10.9-11.0°, 19.9-20.0°, and 24.6-24.7°.

42. A process for preparing a compound of the formula I, or a salt, hydrate or polymorph thereof, 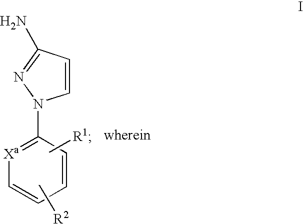
X^{a} is CH, CR¹, CR² or nitrogen;
R¹ and R² are both independently selected from the group consisting of
(1) hydrogen,
(2) halogen,
(3) nitro,
(4) lower alkyl,
(5) halo(lower)alkyl,
(6) hydroxy(lower)alkyl,
(7) cyclo(lower)alkyl,
(8) lower alkenyl,
(9) lower alkoxy,
(10) halo(lower)alkoxy,
(11) lower alkylthio,
(12) carboxyl,
(13) lower alkanoyl,
(14) lower alkoxycarbonyl,
(15) lower alkylene optionally substituted with oxo, and
(16) -Q-Ar², wherein Q is selected from the group consisting of a single bond and a carbonyl, and
wherein Ar² is selected from the group consisting of
(1) aryl, and
(2) heteroaryl,
wherein Ar² is unsubstituted or substituted with a substituent selected from the group consisting of
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
comprising the steps of:
(a) forming a hydrazine solution;
(b) adding a compound of formula V 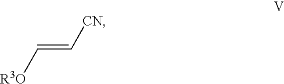
wherein
R³ is selected from the group consisting of
(1) lower alkyl,
(2) aryl, and
(3) —CH₂aryl,
to the hydrazine solution of step (a) to form a mixture; and
(c) heating the mixture of step (b) to a temperature between about 50° C. to about 100° C.;
to afford the compound I, or a salt, hydrate or polymorph thereof.

43. The process of claim 42 wherein the hydrazine solution of step (a) is formed by treating a compound of salt of formula III 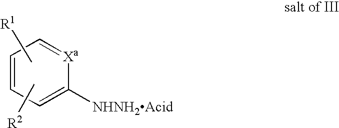
with a base in a solvent.

44. The process of claim 43 wherein the base is potassium tert-butoxide, and the solvent is tert-butanol.

45. The process of claim 42 further comprising the step (e) of treating the compound of formula I 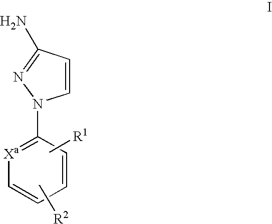
with an acid to form a salt.
